(19) United States
(12) Patent Application Publication
(10) Pub. No.: US 2022/0102001 A1
(43) Pub. Date: Mar. 31, 2022

(54) TUTOR-LESS MACHINE-LEARNING ASSISSTED SHARED DECISION MAKING SYSTEM AND SHARING METHOD THEREOF

(71) Applicant: Yi-Ting LIN, Yunlin County (TW)

(72) Inventor: Yi-Ting LIN, Yunlin County (TW)

(21) Appl. No.: 17/478,972

(22) Filed: Sep. 20, 2021

(30) Foreign Application Priority Data
Sep. 28, 2020 (TW) 109133707

(51) Int. Cl. G16H 50/20; G06N 20/00; G16H 10/60; G16H 10/20; G16H 70/60; G09B 7/06

(52) U.S. Cl. CPC G16H 50/20; G06N 20/00; G09B 7/06; G16H 10/20; G16H 70/60; G16H 10/60

(57) ABSTRACT

A tutor-less machine-learning assisted shared decision making system is provided. The tutor-less machine-learning assisted shared decision making system includes an electronic device having a software component and a cloud server. The software component is installed inside the electronic device and includes a user information component, an information providing component, a user knowledge test component, a user preference component, and a personalized suggestion component of machine-learning model. The user knowledge test component includes a plurality of test questions. A sharing method of a tutor-less machine-learning assisted shared decision making system is also provided. The sharing method of the tutor-less machine-learning assisted shared decision making system includes steps of inputting basic information and clinical information, testing through the user knowledge test component, answering a user preference questionnaire to obtain a prediction result, and transmitting the prediction result to the personalized suggestion component of machine-learning model, and displayed on the user interface.

### CROSS REFERENCE TO RELATED APPLICATIONS

This application claims the priority of Taiwan Patent Application No. 109133707, filed on Sep. 28, 2020, titled “TUTOR-LESS MACHINE-LEARNING ASSISTED SHARED DECISION MAKING SYSTEM AND SHARING METHOD THEREOF”, and the disclosure of which is incorporated herein by reference.

### FIELD OF INVENTION

The present disclosure relates to the technical field of shared decision making system of medical and disease, and particularly, to a tutor-less machine-learning assisted shared decision making system of medical and disease. The present disclosure also relates to a sharing method of tutor-less machine-learning assisted shared decision making system of medical and disease.

### BACKGROUND OF INVENTION

In the process of keeping the body healthy, people must accept many medical procedures and make decisions for each medical procedure. The literature published by Stacey D. et al. (2014) has reported that a duration for making a shared decision of medical and disease is about 25 minutes. Therefore, each decision not only costs the patient financially, but also produces different levels of psychological struggle.

In a shared decision system of medical and disease, a tutor-less shared decision system of medical and disease has many advantages, including expanding a scope of using a shared decision of medical and disease, enhancing the convenience of obtaining the shared decision of medical and disease, being used as a pre-simulation for a tutor-guided shared decision system of medical and disease, saving medical labor costs, allowing the user to have more time to consider and make decisions, and reducing the user's non-patient center influence from the tutor.

A conventional shared decision system of medical and disease is performed by inputting a clinical information and a data of user preference into a machine-learning model to provide suggestions. However, the conventional shared decision system of medical and disease cannot screen users for unguided use.

It is obvious from the above that the conventional technical lacks a shared decision making system of medical and disease that may screen users for unguided use, resulting in the need to rely on the tutor such as a medical staff to complete the shared decision making of medical and disease, which may increase medical labor costs and time-consuming.

Therefore, developing a machine-learning assisted shared decision making system that does not need to be guided by the tutor and a sharing method thereof to immediately provide the decision-making of medical and disease to the user is a problem that needs to be urgently solved in the art.

### SUMMARY OF INVENTION

In order to solve the above-mentioned problem that the conventional technical must rely on tutor to complete the decision-making of medical and disease, an object of the present disclosure is to provide a tutor-less machine-learning assisted shared decision making system. The tutor-less machine-learning assisted shared decision making system provide a user with a test of at least one test question through a user knowledge test component in an electronic device to obtain a test result, so as to understand whether the user has a clear understanding of the relevant knowledge of disease detection, and to screen a qualified user to achieve an object of tutor-less shared decision of medical and disease.

Another object of the present disclosure is to provide a sharing method of tutor-less machine-learning assisted shared decision making system, which provide a user with a test of at least one test question through a user knowledge test component in an electronic device to obtain a test result, so as to understand whether the user has a clear understanding of the relevant knowledge of disease detection, and to screen a qualified user to achieve the object of tutor-less shared decision of medical and disease.

To achieve the objects described above, the present disclosure provides a tutor-less machine-learning assisted shared decision making system. The tutor-less machine-learning assisted shared decision making system comprises an electronic device and a cloud server.

The electronic device comprises a user interface and a software component. The software component is installed inside the electronic device, and the software component comprises a user information component, a user knowledge test component, a user preference component, and a personalized suggestion component of machine-learning model.

The user information component comprises at least one basic information and a clinical data. The basic information and the clinical data are input to the user information component through the user interface. The user knowledge test component comprises at least one test question, and the at least one test question is displayed on the user interface. The user preference component comprises a user preference questionnaire, and the user preference questionnaire is displayed on the user interface. The personalized suggestion component of machine-learning model provides a user with a machine-learning decision-making suggestion through the user interface.

The cloud server is connected to the electronic device via a network. The cloud server comprises a cloud database, a model training and update program, and a machine-learning assisted decision making model and prediction program.

The cloud database is used for storing an information data deriving from the user information component, the user knowledge test component, and the user preference component. The model training and update program is used for updating the information data to obtain an updated information data. The machine-learning assisted decision making model and prediction program receives the information data deriving from the user information component, the user knowledge test component, and the user preference component, and performs computation on the information data or the updated information data to obtain a prediction result. The prediction result is transmitted to the personalized suggestion component of machine-learning model.

In one embodiment, the software component further comprises an information providing component. The information providing component comprises a disease detection related knowledge, and the disease detection related knowledge is displayed on the user interface.

In one embodiment, the information providing component comprises, but is not limited to a video, a text, an image or any combination thereof.

In one embodiment, the clinical data comprises an international prostate symptom score.

In one embodiment, the at least one test question comprises a test question related to a user's understanding of a pros and cons of undergoing a prostate-specific antigen screening and a clinical knowledge and importance of prostate cancer.

In one embodiment, a database serve of the cloud database is provided by a R Shiny server.

In one embodiment, the user preference component comprises a user preference questionnaire, and a reliability of the user preference questionnaire has Cronbach's alpha (Cronbach's a) of 0.838 (based on a physiological aspect) and 0.900 (based on a psychological aspect).

In one embodiment, an algorithm used by the machine-learning assisted decision making model and prediction program comprises multilayer perceptron neural network, random forest, extreme gradient boosting, support vector machine, deep neural network or any combination thereof.

The present disclosure further provides a sharing method of tutor-less machine-learning assisted shared decision making system which comprises steps of:

imputing at least one basic information and a clinical data on a user interface of an electronic device through a user information component in the electronic device, wherein the at least one basic information and the clinical data are transmitted to a machine-learning assisted decision making model and prediction program of a cloud server to perform computation;

performing a test of at least one test question on the user interface through a user knowledge test component in the electronic device to obtain a test result, wherein the test result is transmitted to the machine-learning assisted decision making model and prediction program to perform computation;

answering the at least one test question on the user interface through a user preference component in the electronic device to obtain an answering result, wherein if the answering result is qualified, the qualified answering result is transmitted to the machine-learning assisted decision making model and prediction program to perform computation and obtain a prediction result; and

transmitting the prediction result to a personalized suggestion component of machine-learning model in the electronic device, wherein the prediction result is displayed on the user interface.

In one embodiment, prior to a step of “performing a test of at least one test question on the user interface through a user knowledge test component in the electronic device”, the sharing method further comprises a step of:

providing a disease detection related knowledge through an information providing component in the electronic device, wherein the disease detection related knowledge is displayed on the user interface.

In one embodiment, the sharing method further comprises a step of:

transmitting the at least one basic information and the clinical data, the test result, and the answering result to a cloud database of the cloud server.

In one embodiment, the sharing method further comprises a step of:

transmitting the at least one basic information and the clinical data, the test result, and the answering result to a model training and update program to update an information data and obtain an updated information data, wherein the updated information data is further transmitted to the machine-learning assisted decision making model and prediction program to expand the cloud database.

In one embodiment, a step of “answering the at least one test question on the user interface through a user preference component in the electronic device to obtain an answering result” further comprises a step of:

if the answering result is unqualified, returning to the step of “performing the test of at least one test question on the user interface through the user knowledge test component in the electronic device”.

In one embodiment, a database serve of the cloud database is provided by a R Shiny server.

In one embodiment, the user preference component comprises a user preference questionnaire. A reliability of the user preference questionnaire has Cronbach's alpha (Cronbach's a) of 0.838 (based on a physiological aspect) and 0.900 (based on a psychological aspect).

In one embodiment, the clinical data comprises an international prostate symptom score.

In one embodiment, the at least one test question comprises a test question related to a user's understanding of a pros and cons of undergoing a prostate-specific antigen screening and a clinical knowledge and importance of prostate cancer.

In one embodiment, an algorithm used by the machine-learning assisted decision making model and prediction program comprises multilayer perceptron neural network, random forest, extreme gradient boosting, support vector machine, deep neural network or any combination thereof.

In one embodiment, the information providing component comprises a video, a text, an image or any combination thereof.

A tutor-less machine-learning assisted shared decision making system of the present disclosure and a sharing method of tutor-less machine-learning assisted shared decision making system of the present disclosure provide a user with a test of at least one test question through a user knowledge test component in an electronic device to obtain a test result, so as to understand whether the user has a clear understanding of the relevant knowledge of disease detection, and to screen qualified users to achieve an object of tutor-less shared decision of medical and disease. In addition, a cloud database may be expanded to enhance the accuracy of medical decision making by transmitting information data deriving from a user information component, a user knowledge test component, and a user preference component to the cloud database after the user uses a tutor-less machine-learning assisted shared decision making system.

### BRIEF DESCRIPTION OF DRAWINGS

In order to explain the technical solutions of the present disclosure more clearly, the following will briefly introduce the drawings needed in the description of the embodiments. Obviously, the drawings in the following description are merely some embodiments of the present disclosure. For those skilled in the art, without creative work, other drawings can be obtained based on these drawings.

FIG. 1 is a schematic diagram of a flow chart of a tutor-less machine-learning assisted shared decision making system of the present disclosure.

FIG. 2 is a schematic diagram of a questionnaire of a user information component of the tutor-less machine-learning assisted shared decision making system of the present disclosure.

FIG. 3 is a schematic diagram of a questionnaire of a user knowledge test component of the tutor-less machine-learning assisted shared decision making system of the present disclosure.

FIG. 4A to FIG. 4D are schematic diagrams of questionnaires of a user preference component of the tutor-less machine-learning assisted shared decision making system of the present disclosure.

FIG. 5 is a schematic diagram of a questionnaire of a personalized suggestion component of machine-learning model of the tutor-less machine-learning assisted shared decision making system of the present disclosure.

FIG. 6 is a schematic diagram of a flow chart of a sharing method of tutor-less machine-learning assisted shared decision making system.

### DETAILED DESCRIPTION OF PREFERRED EMBODIMENTS

The following is a specific embodiment to illustrate the implementation of the present disclosure. Those ordinarily skilled in the art can understand the other advantages and effects of the present disclosure from the content disclosed in the present specification. However, the exemplary embodiments disclosed in the present disclosure are for illustrative purposes only and should not be construed as limiting the scope of the present disclosure. In other words, the present disclosure can also be implemented or applied by other different specific embodiments, and various details in the present specification can also be modified and changed based on different viewpoints and applications without departing from the concept of the present disclosure.

The description of the following embodiments refers to the appended drawings to illustrate specific embodiments on which the present disclosure may be implemented.

Unless otherwise stated herein, the singular forms “a” and “the” used in the specification and the appended claims comprise a plurality of entities. Unless otherwise stated herein, the term “or” used in the specification and the appended claims comprises the meaning of “and/or”.

Example: The use of a tutor-less machine-learning assisted shared decision making system to assist a user in making decisions about whether or not to accept the prostate-specific antigen screening for prostate cancer

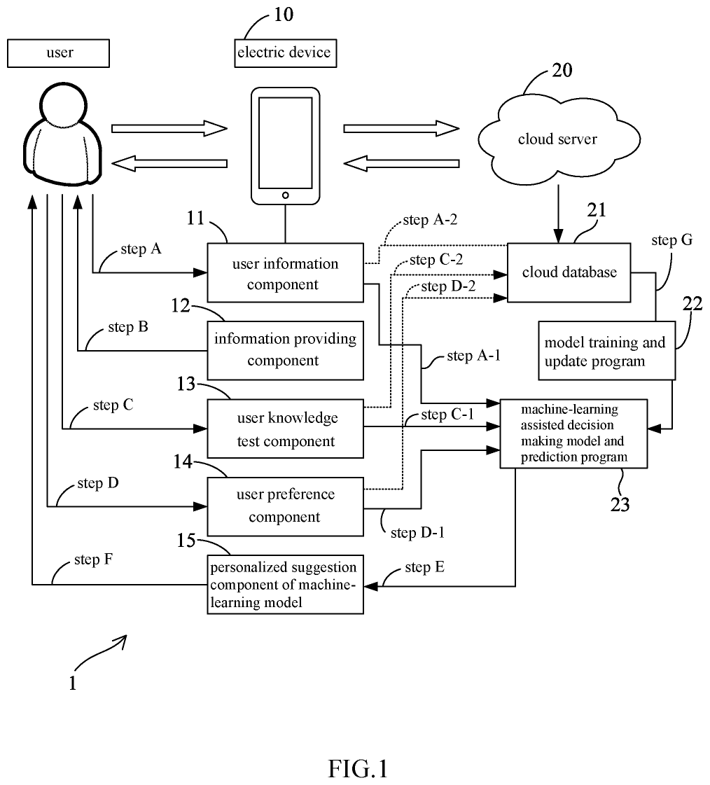

Please refer to FIG. 1. The tutor-less machine-learning assisted shared decision making system 1 comprises an electronic device 10 and a cloud server 20. The electronic device 10 comprises a user interface and a software component. The user interface is used for displaying and inputting information, and the user interface may be a touch display screen. The software component is installed inside the electronic device 10 and the software component includes a user information component 11, an information providing component 12, a user knowledge test component 13, a user preference component 14, and a personalized suggestion component of machine-learning model 15.

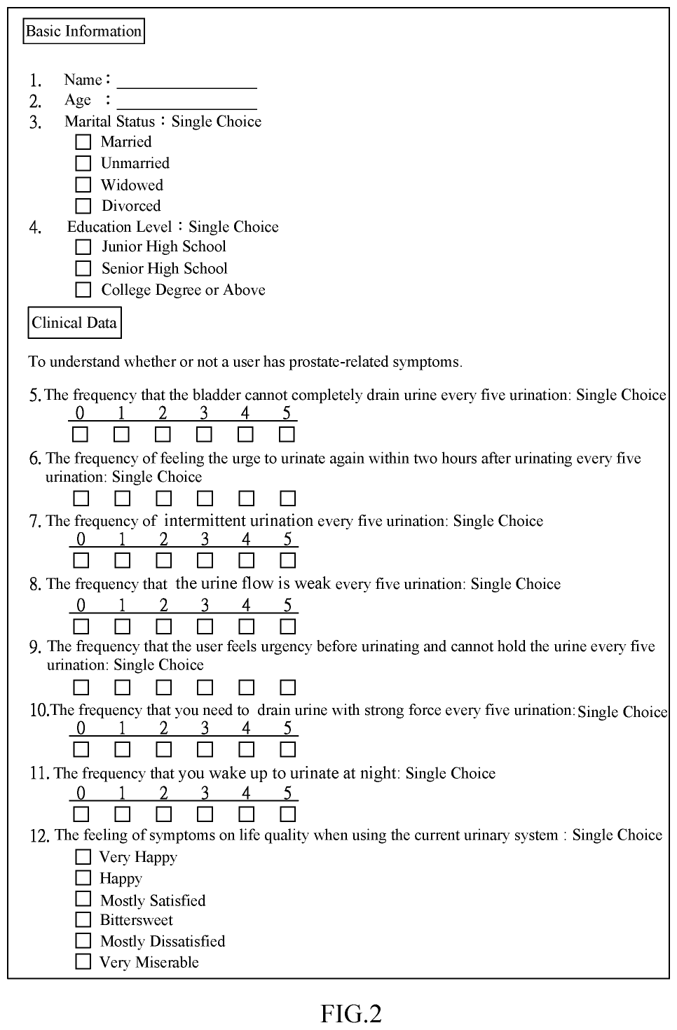

The user information component 11 comprises at least one user's basic information and a clinical data. As shown in items 1 to 4 in FIG. 2, the user's basic information may comprise name, age, marital status, education level, etc. The user's basic information may be input through a user interface or received through the cloud server 20. As shown in items 5 to 12 in FIG. 2, the clinical data may comprise an international prostate symptom score.

The information providing component 12 provides the user with information about a pros and cons of accepting a prostate-specific antigen screening and a clinical knowledge and importance of prostate cancer by displaying a video on the user interface, so that the user may obtain the relevant knowledge of the prostate-specific antigen screening.

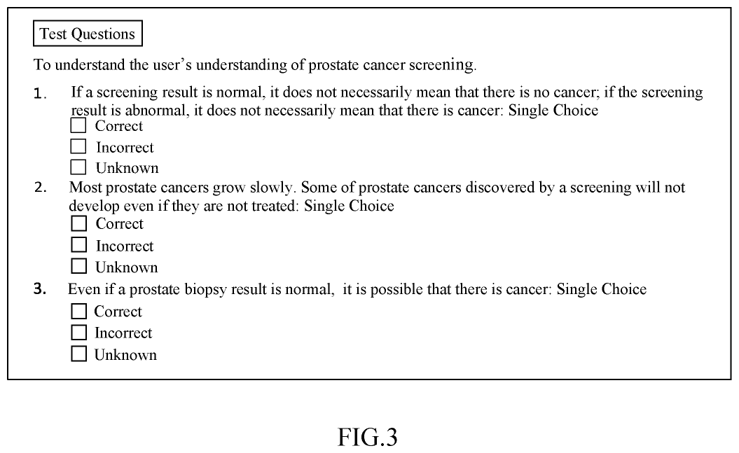

Please refer to FIG. 3. The user knowledge test component 13 comprises a plurality of test questions. The plurality of test questions are used to test the user's understanding of the pros and cons of accepting the prostate-specific antigen screening and the clinical knowledge and importance of prostate cancer, and obtain a test result.

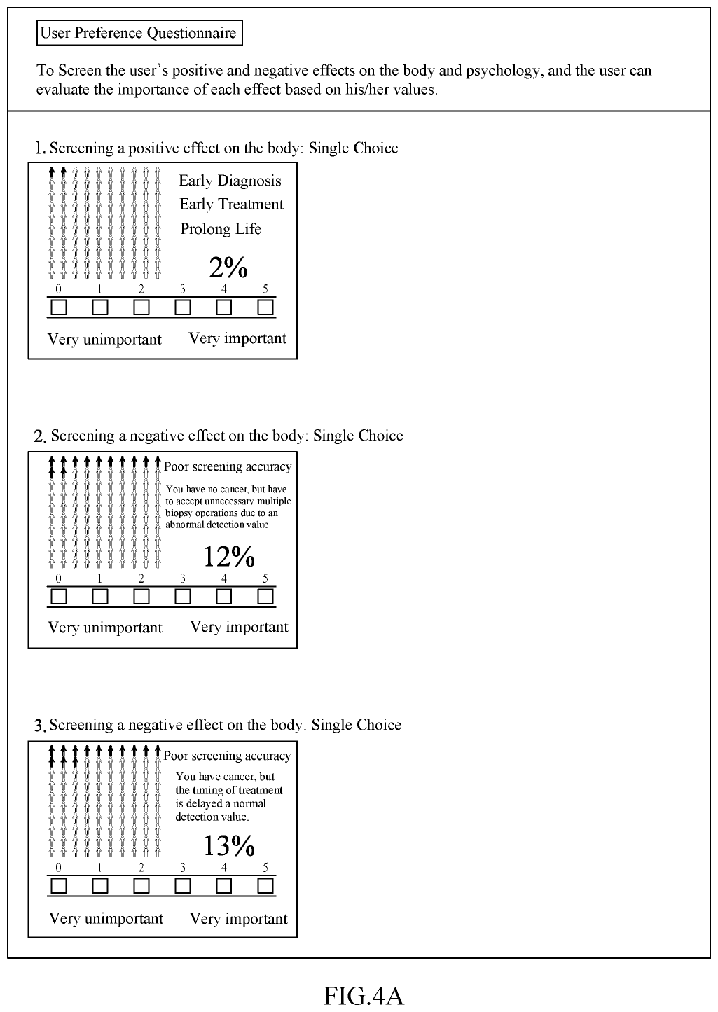

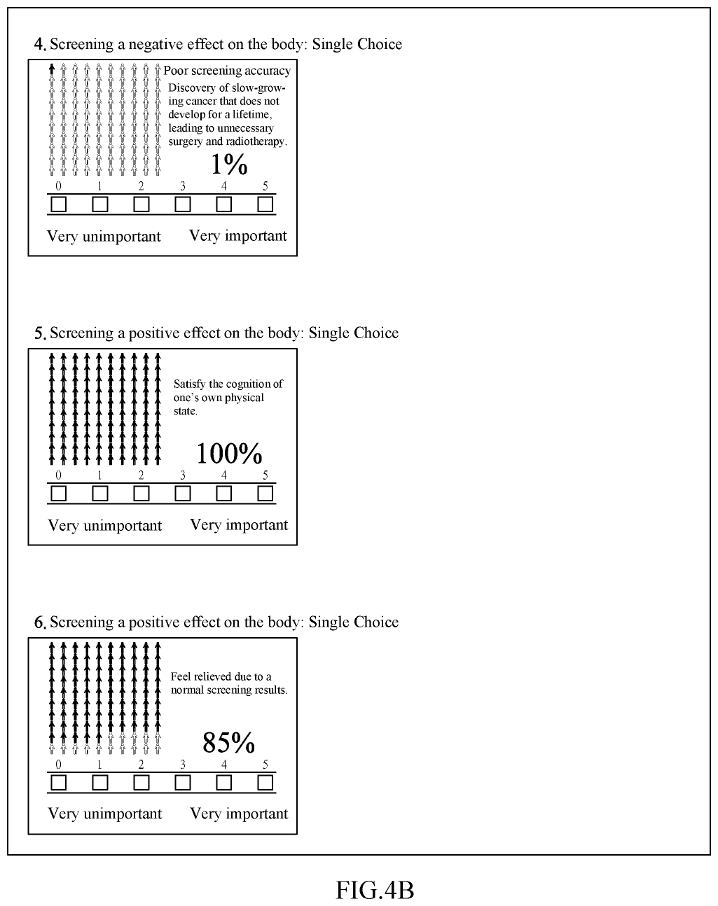

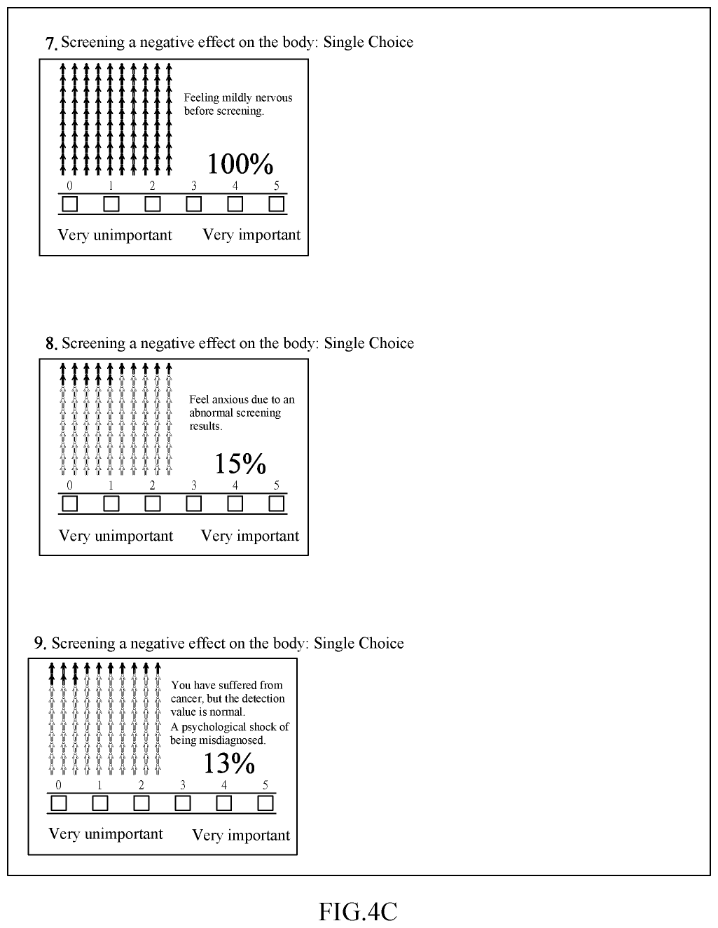

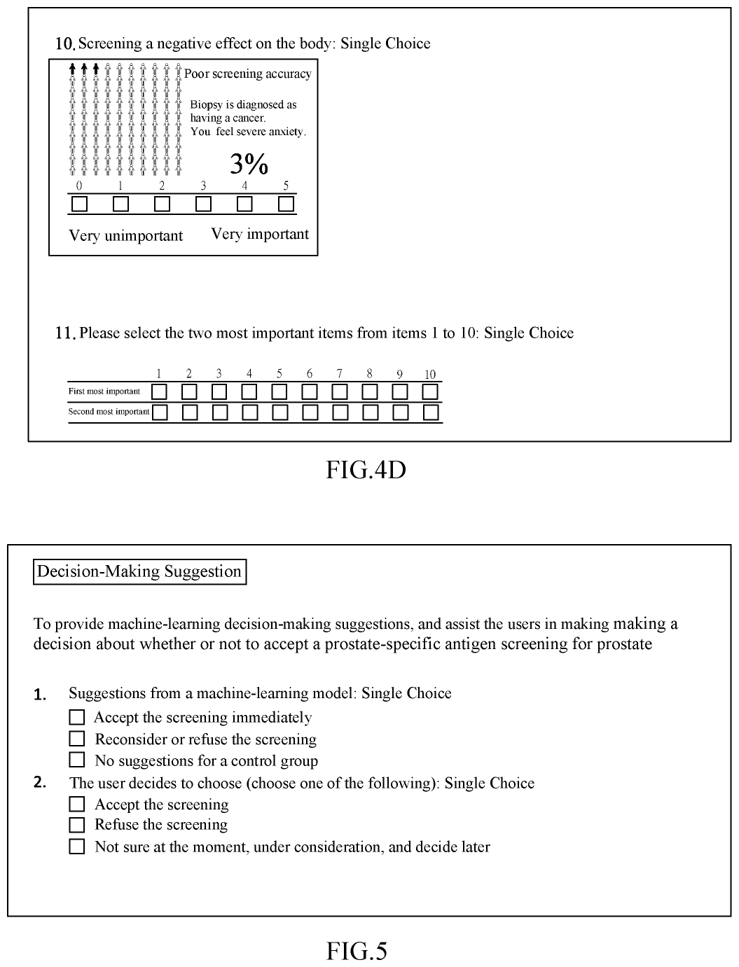

Please refer to FIG. 4A to 4D. The user preference component 14 comprises a user preference questionnaire. The user preference questionnaire is evaluated based on expert validity and questionnaire reliability. Moreover, a reliability of the user preference questionnaire has Cronbach's alpha (Cronbach's a) of 0.838 (based on a physiological aspect) and 0.900 ((based on a psychological aspect).

Please refer to FIG. 5. The personalized suggestion component of machine-learning model 15 provides the user with a machine-learning decision-making suggestions through the user interface and assists the user in making a decision about whether or not to accept the prostate-specific antigen screening for prostate cancer.

Please refer to FIG. 1. The cloud server 20 may be connected to the electronic device 10 via a network. The cloud server 20 comprises a cloud database 21, a model training and update program 22, and a machine-learning assisted decision making model and prediction program 23. In one embodiment, the cloud database 21 is used to anonymously store information data deriving from the user of the user information component 11, the user knowledge test component 13, and the user preference component 14 through a R Shiny server.

In one embodiment, the model training and update program 22 is used to update weekly new information data deriving from the user information component 11, the user knowledge test component 13, and the user preference component 14 through R Shiny server.

In one embodiment, the machine-learning assisted decision making model and prediction program 23 uses the information data deriving from 520 users to build a model and uses a bootstrapping method to perform unbiased data splitting of a modeling group and a test group, followed by obtaining a parameter through an ant lion optimizer method. The parameter is then calculated through an algorithm such as multilayer perceptron (MLP), random forest (RF), extreme gradient boosting (XGboost, XGB), support vector machine (SVM), and deep neural networks (DNN) to generate a model. Finally, the user's information data is brought into the model to obtain a prediction result. The prediction result is transmitted to the personalized suggestion component of machine-learning model 15 of the software component of the electronic device 10 and displayed on the user interface of the electronic device 10, so that the user may obtain the prediction result.

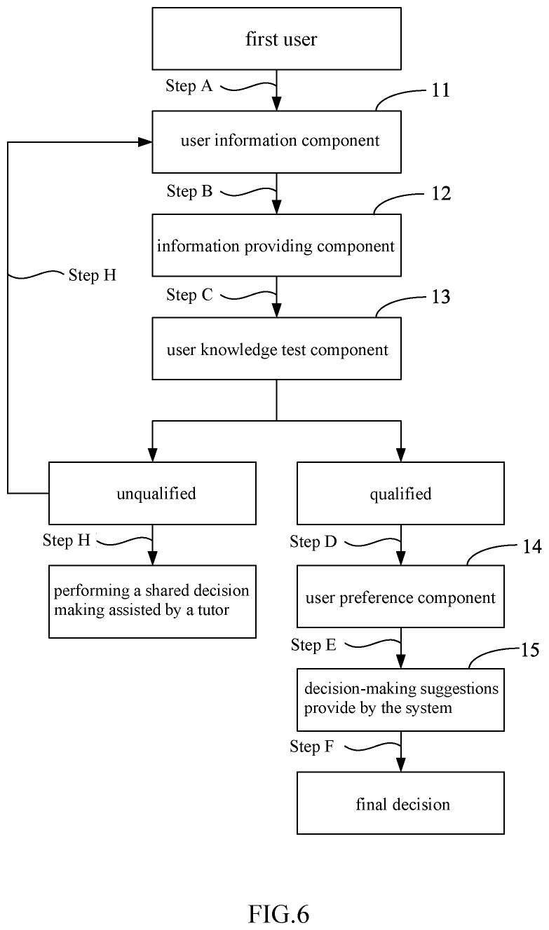

Please refer to FIG. 1 and FIG. 6. A sharing method of tutor-less machine-learning assisted shared decision making system comprises the following steps:

Step A: inputting a basic information and a clinical data of a first user on a user interface through a user information component 11 of an electronic device 10. As shown in step A-1 and step A-2, the basic information and the clinical data are respectively transmitted to a machine-learning assisted decision making model and prediction program 23 for calculation and transmitted to a cloud database 21 to expand a data of the cloud database 21.

Step B: providing the first user with a pros and cons of accepting a prostate-specific antigen screening and a clinical knowledge and importance of prostate cancer through an information providing component 12 of the electronic device 10.

Step C: for the first user, performing a test of understanding of the pros and cons of accepting the prostate-specific antigen screening and the clinical knowledge and importance of prostate cancer on the user interface through a user knowledge test component 13 of the electronic device 10 to obtain a test result. As shown in step C-1 and step C-2, the test result is respectively transmitted to the machine-learning assisted decision making model and prediction

program 23 for calculation and transmitted to the cloud database 21 to expand the data of the cloud database 21.

Step D: based on the test result of step C, for the first user who is qualified, answering a user preference questionnaire on the user interface through a user preference component 14. As shown in step D-1 and step D-2 As shown, an answer result of the user preference questionnaire is respectively transmitted to the machine learning auxiliary decision-making model and prediction program 23 for calculation to obtain a prediction result, and transmitted to the cloud database 21 to expand the data of the cloud database.

Step E: transmitting the prediction result to a personalized suggestion component of machine-learning model 15 of the software component of the electronic device 10 and displaying the prediction result on the user interface of the electronic device 10, so that the first user may obtain the prediction result.

Step F: for the first user, making a decision whether or not to accept the prostate-specific antigen screening for prostate cancer.

Step G: updating an information data of the basic information and the clinical data, the test result, and the answer result of the user preference questionnaire which are transmitted to the cloud database 21 respectively through step A-2, step C-2, and step D-2 by a model training and update program 22 obtain an updated information data of the first user, and transmitting the updated information data to a machine-learning assisted decision making model and prediction program 23 to expand the cloud database.

Step H: based on the test result of step C, for the first user who is unqualified, returning to step B and step C, or performing a shared decision making assisted by a tutor.

When a second user uses the tutor-less machine-learning assisted shared decision making system 1 of the present disclosure, the database of the machine-learning assisted decision making model and prediction program 23 used by the tutor-less machine-learning assisted shared decision making system 1 already comprises the prediction result of the first user. Therefore, the tutor-less machine-learning assisted shared decision making system 1 of the present disclosure may not only provide a medical decision-making suggestions without a guidance of the tutor, but also may expand the cloud database after the user uses the tutor-less machine-learning assisted shared decision making system 1 to enhance the accuracy of medical decision-making suggestions.

The above-mentioned embodiments only exemplarily illustrate the tutor-less machine-learning assisted shared decision making system and the sharing method thereof of the present disclosure, and are not used to limit the present disclosure. Anyone familiar with the technology can modify and change the above-mentioned embodiments without departing from the concept and scope of the present disclosure. Therefore, the claimed scope of the present disclosure should be as stated in the appending claims described below.

What is claimed is:

1. A tutor-less machine-learning assisted shared decision making system, comprising:
an electronic device comprising a user interface and a software component, wherein the software component is installed inside the electronic device, and the software component comprises:
a user information component comprising at least one basic information and a clinical data, wherein the basic information and the clinical data are input to the user information component through the user interface;
a user knowledge test component comprising at least one test question, wherein the at least one test question is displayed on the user interface;
a user preference component comprising a user preference questionnaire, wherein the user preference questionnaire is displayed on the user interface, and
a personalized suggestion component of machine-learning model providing a user with a machine-learning decision-making suggestion through the user interface; and
a cloud server connected to the electronic device via a network, wherein the cloud server comprises:
a cloud database used for storing an information data deriving from the user information component, the user knowledge test component, and the user preference component;
a model training and update program used for updating the information data to obtain an updated information data, and
a machine-learning assisted decision making model and prediction program receiving the information data deriving from the user information component, the user knowledge test component, and the user preference component, and performing computation on the information data or the updated information data to obtain a prediction result, wherein the prediction result is transmitted to the personalized suggestion component of machine-learning model.

2. The tutor-less machine-learning assisted shared decision making system according to claim 1, wherein the software component further comprises an information providing component, and wherein the information providing component comprises a disease detection related knowledge, and the disease detection related knowledge is displayed on the user interface.

3. The tutor-less machine-learning assisted shared decision making system according to claim 2, wherein the information providing component comprises a video, a text, an image or any combination thereof.

4. The tutor-less machine-learning assisted shared decision making system according to claim 1, wherein the clinical data comprises an international prostate symptom score.

5. The tutor-less machine-learning assisted shared decision making system according to claim 4, wherein the at least one test question comprises a test question related to a user's understanding of a pros and cons of undergoing a prostate-specific antigen screening and a clinical knowledge and importance of prostate cancer.

6. The tutor-less machine-learning assisted shared decision making system according to claim 1, wherein a database serve of the cloud database is provided by a R Shiny server.

7. The tutor-less machine-learning assisted shared decision making system according to claim 1, wherein the user preference component comprises a user preference questionnaire, and a reliability of the user preference questionnaire has Cronbach's alpha (Cronbach's a) of 0.838 (based on a physiological aspect) and 0.900 (based on a psychological aspect).

8. The tutor-less machine-learning assisted shared decision making system according to claim 1, wherein an algorithm used by the machine-learning assisted decision making model and prediction program comprises multilayer perceptron neural network, random forest, extreme gradient boosting, support vector machine, deep neural network or any combination thereof.

9. A sharing method of tutor-less machine-learning assisted shared decision making system, comprising steps of:
imputing at least one basic information and a clinical data on a user interface of an electronic device through a user information component in the electronic device, wherein the at least one basic information and the clinical data are transmitted to a machine-learning assisted decision making model and prediction program of a cloud server to perform computation;
performing a test of at least one test question on the user interface through a user knowledge test component in the electronic device to obtain a test result, wherein the test result is transmitted to the machine-learning assisted decision making model and prediction program to perform computation;
answering the at least one test question on the user interface through a user preference component in the electronic device to obtain an answering result, wherein if the answering result is qualified, the qualified answering result is transmitted to the machine-learning assisted decision making model and prediction program to perform computation and obtain a prediction result; and
transmitting the prediction result to a personalized suggestion component of machine-learning model in the electronic device, wherein the prediction result is displayed on the user interface.

10. The sharing method according to claim 9, wherein prior to a step of “performing a test of at least one test question on the user interface through a user knowledge test component in the electronic device”, the sharing method further comprises a step of:
providing a disease detection related knowledge through an information providing component in the electronic device, wherein the disease detection related knowledge is displayed on the user interface.

11. The sharing method according to claim 9, wherein the sharing method further comprises a step of:
transmitting the at least one basic information and the clinical data, the test result, and the answering result to a cloud database of the cloud server.

12. The sharing method according to claim 11, wherein the sharing method further comprises a step of:
transmitting the at least one basic information and the clinical data, the test result, and the answering result to a model training and update program to update an information data and obtain an updated information data, wherein the updated information data is further transmitted to the machine-learning assisted decision making model and prediction program to expand the cloud database.

13. The sharing method according to claim 11, wherein a step of “answering the at least one test question on the user interface through a user preference component in the electronic device to obtain an answering result” further comprises a step of:
if the answering result is unqualified, returning to the step of “performing the test of at least one test question on the user interface through the user knowledge test component in the electronic device”.

14. The sharing method according to claim 11, wherein a database serve of the cloud database is provided by a R Shiny server.

15. The sharing method according to claim 9, wherein the user preference component comprises a user preference questionnaire, and a reliability of the user preference questionnaire has Cronbach's alpha (Cronbach's a) of 0.838 (based on a physiological aspect) and 0.900 (based on a psychological aspect).

16. The sharing method according to claim 9, wherein the clinical data comprises an international prostate symptom score.

17. The sharing method according to claim 16, wherein the at least one test question comprises a test question related to a user's understanding of a pros and cons of undergoing a prostate-specific antigen screening and a clinical knowledge and importance of prostate cancer.

18. The sharing method according to claim 9, wherein an algorithm used by the machine-learning assisted decision making model and prediction program comprises multilayer perceptron neural network, random forest, extreme gradient boosting, support vector machine, deep neural network or any combination thereof.

19. The sharing method according to claim 10, wherein the information providing component comprises a video, a text, an image or any combination thereof.
